(19) United States
(12) Patent Application Publication
(10) Pub. No.: US 2024/0025096 A1
(43) Pub. Date: Jan. 25, 2024

(54) IMPROVEMENTS RELATING TO FACE MASKS

(71) Applicant: INTERSURGICAL AG, Vaduz (LI)

(72) Inventors: Matthew James William LEARY, Wokingham, Berkshire (GB); David Simon BOTTOM, Wokingham, Berkshire (GB); Andrew Neil MILLER, Wokingham, Berkshire (GB); Simon Robert PAYNE, Wokingham, Berkshire (GB)

(21) Appl. No.: 18/256,566

(22) PCT Filed: Dec. 9, 2021
(86) PCT No.: PCT/EP2021/085087

§ 371 Date: Jun. 8, 2023

(30) Foreign Application Priority Data
Dec. 9, 2020 (GB) 2019406.4
Jul. 16, 2021 (GB) 2110291.8

(51) Int. Cl. A41D 13/11; B29C 45/17; B29C 45/14; A62B 23/02

(52) U.S. Cl. CPC A41D 13/1146; B29C 45/1704; B29C 45/14336; A62B 23/025; B29C 2045/14459; B29C 2045/1725

(57) ABSTRACT

Face masks, including filter masks, and methods for their production. The disclosed methods include using an overmoulding step to both form a sealing member and to fix the sealing member to a support frame and a sheet of filtering material.

The present invention relates to face masks, to methods of manufacturing face masks, and to face masks produced by the disclosed methods.

Face masks are used for a range of reasons including therapy, and for personal protection (in both medical and non-medical settings).

Some masks are required to filter out particulate substances from the air, ie filter masks. Such masks are typically one of two types. In particular, the face mask may have a mask body that itself functions to filter out particulate substances from the air, and this type of filter mask is typically lightweight and relatively inexpensive, so as to be single-use and disposable. Alternatively, the face mask may have a mask body adapted to receive a filter cartridge. Such a filter cartridge may be replaceable, so that the face mask is reusable.

Filter masks may be worn in work or public environments to protect from dust, smoke and other noxious substances, and to protect from pathogens, such as viruses or bacteria. In a healthcare environment, for example, filter masks are used to prevent the inhalation of pathogens from the environment by preventing the passage of particulates, eg droplets and aerosols, through the mask. Filter masks may also protect others from exhalation of pathogens by the wearer.

Filter masks typically comprise a filter, such as a sheet of textile or nonwoven filter 25 material, and a means for retaining the mask on the wearer's head, for example by loops of elastic which are placed around the ears. In use, the filter covers the user's mouth and nose, or air is otherwise directed through the filter, so that any air inhaled by the wearer passes through the filter. In order to protect others from exhalation of pathogens by the wearer, the filter may be arranged so that any air 30 exhaled by the wearer passes through the filter.

Filter masks worn in work environments, such as by healthcare professionals, must stay securely fitted to the face and be comfortable to wear for a substantial period of time in an environment in which the risk of contamination may be high. Such masks must closely fit the wearer's face, and preferably form a seal against the face so that inhaled and exhaled air cannot pass around the edge of the mask.

Filter masks are commonly single-use products, produced and sold in high volume. For products of this type to be commercially viable, the cost and complexity of manufacture must be kept to a minimum.

In many circumstances it is desirable that the mask seals to the wearer's face, to prevent the ingress or egress of air about the periphery of the mask. Creating an effective seal against the varying contours of the face can be challenging, and cushion seals formed of a resilient material that compresses against the face to form a seal are commonly employed. However, such seals can become dislodged during movement of the wearer's face (eg during speech), and may be prone to lateral displacement across the face.

There has now been developed a mask and a method for its production which overcomes or substantially mitigates the above mentioned and/or other problems associated with the prior art.

According to a first aspect of the invention there is provided a method of manufacturing a filter mask, the filter mask comprising a support frame, a filter and a sealing member, wherein the method comprises:
a) locating the filter and the support frame in a mould; and
b) injecting a polymeric material into the mould to form the sealing member,
   wherein the sealing member is brought into engagement with the filter and the support frame, during injection moulding of the sealing member, in a manner that fixes the sealing member to the support frame and the sheet of filtering material.

The injection moulding of the sealing member in the method according to the invention may be overmoulding. By “overmoulding” is meant, in the context of the invention, an injection moulding process in which a first material is moulded onto a second material to form a single item, and where the first material may partially or wholly cover the second material.

The method of manufacturing a filter mask according to the first aspect of the invention is advantageous principally because the step of injection moulding of the sealing member fixes the sealing member to the support frame and the sheet of filtering material, which may therefore fix the relative positions of these components without requiring any further assembly or moulding steps. Face masks, in particular filter masks, are products which are produced at high volume and sold at low cost. As the profit margin on each individual item is low, reducing the cost and complexity of the manufacturing method is key to producing a viable product.

According to a further aspect of the invention, there is provided a filter mask manufactured according to the method described above. According to a further aspect of the invention, there is provided a filter mask comprising a support frame, a filter and a sealing member, wherein the sealing member is fixed to the support frame and the filter.

The support frame and the filter may together define a mask body, which forms a cavity for the accommodation of the wearer's mouth and nose. The mask body may be curved in at least the transverse plane.

The support frame may be made in a preceding step of the method of the invention. The support frame may be made in a preceding step by injection moulding. Thus, the method may further comprise a preceding step in which the support frame is formed by injection moulding. The method may further comprise a step of transferring the support frame from a first mould in which the support frame is injection moulded to a second mould in which the sealing member is injection moulded.

The support frame may be made of a plastics material, such as polypropylene, poly(styrene-butadiene-styrene) (SBS), polycarbonate, plastomers, thermoplastics, thermoplastic elastomers, thermosets or blends or combinations of the foregoing.

The support frame may have an external face and an internal face, the internal face, in use, facing the wearer and the external face, in use, facing away from the wearer.

The support frame may support at least a portion of the filter, eg by preventing distortion of the sheet of filtration material. The support frame may provide rigidity to the mask. The support frame may retain the filter media at a distance from the face, thus preventing the filter media from coming into direct contact with the mouth and/or nose and reducing or preventing degradation and contamination of the filter, and increasing comfort for the wearer.

The support frame may take the form of a rim, for example that matches the shape of the filtering material. The rim may have the form of a closed loop. The rim may define an aperture through which inhaled and/or exhaled gases flow, in use. When the filter is placed onto the support frame, the support frame may extend about the periphery of the filter. The support frame may comprise a shoulder extending from an inner edge of the rim, providing a surface on which the filter can be located, facilitating retention of the filter in the mould prior to injection moulding of the sealing member. The filter may be located on the internal face of the support frame. The periphery of the filter may be sealed against the support frame such that air cannot flow between an edge of the filter and the support frame; inhaled air must pass through the filter.

Alternatively, or additionally to the rim, the support frame may comprise one or more ribs, eg a plurality of ribs, which are configured to abut against a surface of the filter, for providing support to the filter and rigidity to the mask. The one or more ribs may have a shape which corresponds to the shape of a surface of the filter. The one or more ribs may be shaped such that they extend across a surface of the filter. The one or more ribs may extend between two opposing sides of the rim or support frame, and be shaped to abut a surface of the filter. The one or more ribs may have an arcuate shape. Such ribs may provide structure and/or rigidity to the rim or support frame, helping to maintain the shape of the mask and prevent distortion of the filter material.

The external face of the support frame may further comprise a shield which, in a filter mask according to the invention, extends over at least part of a surface of the filter. The shield may be separated from the filter, and may be positioned in front of the filter, such that air may flow between the shield and the filter, eg before the air is drawn through the filter by the wearer during inhalation. There may therefore be a space or void between the shield and the filter. An inlet aperture, through which air may be drawn by a wearer inhaling, may be defined between an edge of the shield and a surface of the filter. The shield may not contact the filter.

The shield may provide a barrier upon which particles in the environment impinge. The shield may therefore reduce the quantity of particles incident on the filter during use, and hence may extend the effective life of the filter and hence of the filter mask. The separation between the shield and the filter enables incoming air to flow through the filter across its entire surface, even where the shield is impermeable or substantially impermeable. The shield may additionally provide reinforcement to the mask, preventing longitudinal collapse of the mask, and keeping it in the correct place relative to the wearer's nose and chin.

The shield may be formed of plastics material. This may form a solid barrier to deflect particulates, and reduce the risk of physical damage to the filter by providing a protective barrier. The shield may form a rigid barrier.

The shield may alternatively be a composite shield formed of two or more components. The composite shield may comprise a sheet of filter material (the shield filter) and a shield frame, the shield filter overlaying and being affixed to the shield frame. The shield frame may be a plastic frame, and may form part of the support frame. The shield frame may comprise a shield rim substantially the same shape as the intended shield and/or the shield filter, the shield rim being affixed to the periphery of the shield filter. Such a composite shield actively absorbs and blocks contaminants, and may reduce the build-up of humidity in the mask, increasing comfort for the wearer. The composite shield may be removably fixed to the mask, such that it can be replaced if it becomes saturated; either the entire composite shield may be removeable and replaceable, or just the sheet of filter material may be replaceable.

In the following paragraphs, unless indicated otherwise references to the “shield” refer both to a shield formed of a plastics material, and to a composite shield formed of a shield frame and shield filter. In addition, unless indicated otherwise references to the “filter” refer to the main filter, that is, the filter retained by the support frame, and references to the “shield filter” refer to a filter forming part of a composite shield.

The surface area of the shield may be at least 50%, at least 60%, at least 70%, or at least 80%, or at least 90%, or at least 95% of the surface area of the filter. The surface area of the shield may be up to 110% of the surface area of the filter, or up to 105%, or up to 100%, or up to 95% of the surface area of the filter. The shield may extend over at least 50%, at least 60%, at least 70%, or at least 80%, or at least 90%, or at least 95%, or up to 100% of the surface area of the filter.

The shield may extend over the entire surface area of the filter, or may extend beyond the periphery of the filter such that the shield overhangs the edges of the filter. Alternatively, the shield may extend over less than 100% of the surface area of the filter, such that a portion of the filter is exposed at the periphery of the shield. Alternatively, or additionally, the shield may have apertures formed therein through which the sheet of filtering material is exposed, which may assist the flow of air and help to evenly distribute the particle load on the filter. The shield may alternatively have no apertures, but form a continuous barrier. Thus, the shield may comprise a continuous surface that extends across a majority of the surface of the filtering material.

The support frame may further comprise one or more seal supporting members. The support frame may comprise one or more seal supporting members located at any position at which the seal requires reinforcement. The support frame may comprise one or more seal supporting members in the chin region, the nasal region and/or the cheek region. In particular, the support frame may comprise one or more seal supporting members in the chin region. The one or more seal supporting members provide additional support to the seal and help to prevent collapse of the seal and improve fit. The one or more seal supporting members may extend outwardly from the support frame, abutting the sealing member, and have a shape corresponding to the shape of the portion of the sealing member to which it abuts. The one or more seal supporting members may have an arcuate shape.

At least one seal supporting member may be located in the chin region, and the chin region of the support frame may comprise at least one downwardly depending seal supporting member. The at least one seal supporting member may comprise a tab extending outwardly from the support frame, and may extend downwardly in a direction substantially perpendicular to the support frame. The at least one seal supporting member may extend to the distal edge of the sealing member, or may only extend across a portion of the depth of the sealing member. The at least one seal supporting member may be bonded to the seal, and/or the seal may be overmoulded to the seal supporting member during the overmoulding process.

The seal supporting member provides additional reinforcement in the chin region, helping to prevent collapse of the seal in this region. This helps to improve and maintain the seal.

The support frame is typically formed of a plastics material that retains its shape in use. The support frame may comprise a rim and/or a shield or shield frame and/or one or more connection members and/or one or more seal supporting members. Where multiple components are present the entire support frame may be integrally formed, eg by injection moulding, or the support frame may be formed in two or more pieces which are connected together eg by gluing, welding or mechanical fastening. Thus, where the support frame comprises a rim and a shield or shield frame, the rim and shield or shield frame may be integrally formed in one injection moulding step, or the rim and the shield or shield frame may be formed separately before being bonded together either chemically or mechanically.

The filter may be formed from a filter substrate that is sufficiently dense to prevent the passage of a majority of airborne particles, such as dust particles, droplets and aerosols. The mask of the present invention may filter out at least 80%, or at least 85%, or at least 90%, or at least 95% or at least 99% of airborne particles.

In countries including the UK and those in the European Union, the FFP standards specify requirements for filtering masks as respiratory protective devices. The EN 149 standard defines three classes of filter efficiency, namely FFP1, FFP2 and FFP3. The mask of the present invention may be a FFP3 mask, for example, which under the EN 149 standard filters out at least 99% of airborne particles at 95 L/minute airflow, and with a total inward leakage of less than 2%. The mask of the present invention may be a FFP2 mask, which filters out at least 94% of airborne particles at 95 L/minute, and with a total inward leakage of less than 8%. In the USA, the U.S. National Institute for Occupational Safety and Health (NIOSH) provides an N95 classification of air filtration, and the mask of the present invention may be an N95 mask, meaning that it filters at least 95% of airborne particles.

Materials suitable for use as the filter substrate are known in the art, and any suitable material may be used for the sheet of filtering material in the present invention. For example, the filter may be a nonwoven, electrostatic, meltblown, spunbond, textile, nanofiber or nanoweb material, or be formed of a glass fibre media. The filter may comprise a plurality of layers to enhance its performance and increase comfort for the wearer. Thus, the filter may comprise one or more layers selected from nonwoven, electrostatic, meltblown, spunbond, textile, nanoweb and/or nanofiber material. For example, the filter may comprise layers of nonwoven, electrostatic and meltblown material, together with a textile layer to increase comfort for the wearer and provide structure to the filter. The filter may be in any appropriate configuration, for example the surface of the filter may be smooth, eg it may be flat or planar, or the filter may be pleated. The filter (pleated or non-pleated) may have a flat or planar region about its periphery to facilitate the overmoulding process. The filter may further comprise one or more additives to enhance its effectiveness in reducing or preventing the transmission of pathogens, such as antimicrobial agents.

The filter that is located in the mould in the method of the present invention may comprise the filter substrate only, and may be flexible in form. The filter may include no support for the filter substrate until fixed to the support frame and the sealing member.

The filter may be cut to the correct shape from a larger sheet of filter substrate, for example using a die. Where the filter is formed from a plurality of layers, the filter may be cut to size and the edges sealed in a single hot stamp process, in which heat is used to seal the peripheral edges of the filter during the cutting process. The sealed periphery, in use, prevents ingress of moisture or contaminants to the interior of the filter material, between the layers. The size and shape of the filter may correspond to the size and shape of at least a portion of the support frame. For example, the size and shape of the filter may correspond to the size and shape of the rim of the support frame, such that the filter can be positioned on the rim, eg on a shoulder inwardly extending from the rim, such that the support frame extends about the periphery of the filter.

In some embodiments, the filter mask may comprise a single filter. In some embodiments, the filter mask may comprise two, or more filters. The filter mask may comprise a first filter at a first side of the mask, and a second filter at a second side of the mask. Each of the first and second filters may be brought into contact with the support frame, before being fixed to both the support frame and the sealing member by the overmoulding of the sealing member. In this embodiment, the support frame may comprise a housing having first and second apertures, in which the first and second filters may be located. The size and shape of each filter may correspond to the size and shape of the relevant aperture in the support frame, such that the filter can be positioned on the support frame, eg on a shoulder inwardly extending from a periphery of the aperture, such that the support frame extends about the periphery of each filter.

In some embodiments, the filter(s) may be brought into contact with the support frame before the support frame and filter(s) are together located in the mould. For example, the filter(s) may be located on the rim of the support frame before the support frame and filter(s) are together located in the mould. Alternatively, the support frame may be placed into the mould before the filter(s) is positioned on the support frame. In either case, both the support frame and the filter(s) are located in the mould prior to injection moulding of the seal member. Preferably, the support frame and the sheet(s) of filtering material are not fixed or bonded together prior to locating in the mould.

The sealing member is adapted to fit snugly against the wearer's face, and is overmoulded about both the filter and the support frame. Thus, the filter and the support frame are located in the mould, and their relative positions are fixed by injection moulding of the sealing member. This fixes the support frame, filter and sealing member together in a single step, without the need for additional adhesive or overmoulding steps, thereby reducing time and costs.

To fix the filter and support frame together in a single overmoulding step, the filter may be positioned on the external or internal face of the support frame. The filter may be positioned on the rim of the support frame, such that the edges of the sheet of filtering material and support frame are substantially aligned. The filter may be positioned on a shoulder extending inwardly from a rim of the support frame. During injection of the polymeric material that forms the sealing member, a portion of the polymeric material may envelop the edges of the sheet of filtering material and the support frame. Thus, the peripheral edge of the filter and an outer or inner edge of the support frame, rim and/or shoulder of the support frame, may be enclosed within a portion of the polymeric material that forms the sealing member.

Alternatively, during overmoulding of the seal the injected material may be directed between the filter and a portion of the support frame (eg a rim of the support frame, a shoulder extending inwardly from a rim of the support frame, an outer edge of the support frame or an inner edge of the support frame), such that the injected material forms an adhesive layer between the filter and support frame about the periphery of the filter. The periphery of the filter may be enveloped by the injected material, which then bonds to the support frame (eg to the rim of the support frame). The injected material may be absorbed into the filter about its periphery, increasing the bond between the seal and the filter and providing an additional protection against ingress of moisture between layers of the filter material.

By “substantially aligned” is meant that the edges of the filtering material and support frame are located close enough together that they can be enveloped or otherwise adhered together by a portion of the sealing member.

The sealing member may be formed of an elastomeric material, and suitable materials for use in making sealing members for face masks are known in the art. For example, the sealing member may be formed of a thermoplastic elastomer (TPE) or a thermoset elastomer, such as liquid silicone rubber.

The sealing member may be shaped to fit snugly and to seal against the wearer's face, such that all of the air inhaled by the wearer passes through the filtering material. To increase comfort and sealing effectiveness, the sealing member may be in the form of a cushion. That is, the sealing member may form a resiliently compressible region between the support frame and the wearer's face, increasing comfort and improving the seal.

Additives may be used to increase the effectiveness of the mask at preventing the passage of pathogens. According to a further aspect of the invention there is provided a filter mask comprising a support frame, a filter and a sealing member, wherein the sealing member is fixed to the support frame and the filter, and wherein at least a portion of the filter mask comprises one or more additives selected from antibacterial agents, antimicrobial agents, antiviral agents and antifungal agents.

Any aspect of the support frame and/or seal and/or filter may comprise one or more additives to help increase the effectiveness of the mask in reducing or preventing the spread of bacteria or other pathogens, including but not limited to antibacterial agents, antimicrobial agents, antiviral agents and/or antifungal agents. In particular, an additive containing silver ions (for example in the form of Silver Knight™) may be incorporated into the plastic of the support frame and/or seal. Silver ions have been found to catalyse the deactivation of pathogenic bacteria and prevent their proliferation, reducing microbial growth within and on the surface of the mask.

The sealing member and, therefore, the face mask, may also extend laterally across the wearer's cheeks, such that a seal is formed between the mask seal and the tissue of the cheeks, and the internal cavity of the mask extends over the cheeks.

During speech, the majority of motion in the face is in the jaw, including vertical separation of the nose and chin, while there is a relatively small amount of movement in the cheeks. In addition, the tissue of the cheeks is softer and more compliant than the tissue of either the nose or chin. This means that a lower degree of flexibility is required in the sealing member to achieve an effective seal with the cheek tissue than with other parts of the face. In addition, sealing in a position further away from the mouth and its movement helps to provide stability and maintain a constant seal.

The sealing member may extend about the periphery of the mask, such that the sealing member comprises corresponding cheek portions which extend laterally across the wearer's cheeks.

It has been found that, when the mask body extends laterally across the wearer's cheeks, the additional lateral stability provided makes it easier for the user to position the mask correctly, and helps to prevent the mask from slipping to either side during use. This lateral stability, and the effective seal produced, makes this mask body and sealing formation suitable for use in other types of mask, including gas delivery masks, and gas delivery filter masks (eg oxygen or aerosol delivery).

The cheek portions may extend laterally across the wearer's cheeks between the upper edges of the malar bones and the lower edge of the mandible of the wearer. The laterally extended cheek portions of the mask may form the part of the mask having the longest dimension in the horizontal direction (ie widthwise, running from a first side edge of the mask to an opposing side edge of the mask). The distance between a first side edge of the mask body and an opposing side edge of the mask body, measured along the surface of the mask body, may be between 1.2 and 2 times, or between 1.2 and 1.8 times, or between 1.3 and 1.7 times, or between 1.4 and 1.6 times larger than the distance between the top edge of the mask body in the nasal region and the bottom edge of the mask body in the chin region, measured along the surface of the mask body.

The first side edge and second side edge of the mask, located in the cheek regions, may comprise a straight or substantially straight or linear edge. The first and second side edges may not be arcuate.

The cheek portions may each have a first edge that extends from a left- or right-side edge of the nasal portion, such that the first edge of the cheek portion is accommodated over the malar bone of the wearer. The first edge may extend along an arcuate path. The first edge may be a concave exterior edge. The transition between the first edge of the cheek portion and the left- or right-side edge of the nasal portion may be arcuate in form.

The transition between the first edge (between the nasal portion and the cheek portion) and the first and/or second side edge (in the cheek portion) may be angular. The transition between the first edge and the first and/or second side edge may be at an angle of between 70 and 110 degrees, or between 80 and 100 degrees, or about 90 degrees.

The first side edge may connect the first and second edges at a distal end of the cheek portion (eg proximate to the wearer's ear). The second side edge may connect the first and second edges at a distal end of the opposing cheek portion (eg proximate to the wearer's other ear).

The cheek portions may each have a second edge that extends from a left- or right-side edge of the mouth portion, such that the second edge of the cheek portion is accommodated above the lower edge of the mandible of the wearer. The second edge may extend along an arcuate path. The second edge may be a concave exterior edge. The transition between the second edge of the cheek portion and the left- or right-side edge of the mouth portion may be arcuate in form.

The transition between the second edge (between the mouth portion and the cheek portion) and the first and/or second side edge (in the cheek portion) may be angular. The transition between the second edge and the first and/or second side edge may be at an angle of between 70 and 110 degrees, or between 80 and 100 degrees, or about 90 degrees.

The tangential angle between a tangent line to the first and/or second edges of each cheek portion and the x-axis may decrease with increasing lateral position. The angle between tangent lines to the first and second edges of each cheek portion may decrease with increasing lateral position. At a distal end of the cheek portion, eg proximate to the wearer's ear, the first and second edges may be substantially parallel. At a distal end of the cheek portion, the first edge may be substantially horizontal and/or linear (extending, in use, in a direction widthwise across the wearer's face), while the second edge is arcuate or substantially arcuate. The first and/or second side edge of each of the cheek portions may be generally linear, and may be aligned with the longitudinal axis of the mask (ie the y-axis). A distal region of each cheek portion may be generally rectangular in shape. This may increase the surface area of the mask body that is covering the wearer's cheeks.

The mask body may extend a greater distance in a transverse direction across the face of the wearer, relative to the distance the mask body extends in the longitudinal direction across the face of the wearer. The greater distance may be relative to the surface of the face of the wearer, ie the distances may be dimensions along the surface of the face, as opposed to linear dimensions.

The mask body may extend a greater distance in a transverse direction, relative to the distance the mask body extends in a longitudinal direction. The greater distance may be relative to the surface of the mask, ie the distances may be dimensions along or parallel to the surface of the mask, as opposed to linear dimensions.

The nasal and chin portions of the sealing member may seal against or about the nose and chin, while the cheek portions extend laterally across and seal against the wearer's cheeks. The chin portion may seal against the front of the wearer's chin, or may seal under the chin. The chin portion of the sealing member may not extend under the mandible. The sealing member may not extend about the wearer's eyes and/or forehead.

The depth of the sealing member in the cheek portion may be smaller than the depth of the sealing member in the nasal portion. The depth of the mask body in the cheek portion may be smaller than the depth of the mask body in the nasal portion. Thus, the portion of the internal cavity formed around the cheeks by the cheek portion of the face mask may be shallower than the portion of the internal cavity formed around the nose and mouth by the nasal and mouth portions of the face mask. The portion of the internal cavity formed around the cheeks by the cheek portions of the face mask may have a smaller internal volume than the portion of the internal cavity formed around the nose and mouth by the nose and mouth regions of the face mask.

The sealing member in the cheek region may comprise a first portion and a second portion, the first portion extending rearwardly, in use, from a peripheral edge of the support frame. The first portion may comprise a proximal edge, which is fixed to the support frame, and a distal edge. The second portion may comprise an outwardly depending lip, extending from the distal edge of the first portion. The second portion may define a face contacting surface. The outwardly depending lip of the second portion may extend between 1 and 10 mm, or between 2 and 6 mm, from the distal edge of the first portion. The thickness of the material forming the first portion may be greater than the thickness of the material forming the second portion. It has been found that the incorporation of an outwardly depending lip in the cheek region helps to form an effective seal in this region, and to retain the seal even during facial movement.

At least a portion of the sealing member in the chin region may comprise a first portion and a second portion, the first portion extending rearwardly, in use, from a peripheral edge of the support frame. The first portion may comprise a proximal edge, which is fixed to the support frame, and a distal edge. The depth of the first portion may vary along its length. The second portion may comprise both an inwardly and an outwardly depending lip, wherein the second portion is fixed to the distal edge of the first portion. The second portion may define a face contacting surface. The outwardly depending lip of the second portion may extend between 1 and 20 mm, or between 5 and 20 mm, from the distal edge of the first portion. The inwardly depending lip of the second portion may extend between 1 and 15 mm, or between 5 and 15 mm, from the distal edge of the first portion. The thickness of the material forming the first portion may be greater than the thickness of the material forming the second portion.

The portions of the mask body which accommodate the wearer's nose and mouth may have an exterior surface with a generally arcuate shape, eg in the transverse plane, while the portion which accommodates the wearer's cheeks may have an exterior surface that is substantially more planar in shape, eg in the transverse plane. The face mask, and the internal cavity of the face mask, may extend laterally across the wearer's cheeks, such that an edge of the mask is situated, in use, within 5 cm, or within 4 cm, or within 3 cm, or within 2 cm of the wearer's ears. The face mask may extend laterally across the wearer's cheeks, such that an edge or a corner of the mask is situated, in use, on or about the zygomatic bone, or on or about the zygomatic process. Thus, the mask may substantially cover the wearer's cheeks.

The mask body may comprise a side wall extending around at least a portion of its periphery. The side wall may form part of the support frame. The side wall may have a greater depth in the nasal region than in the cheek regions, in order to accommodate the wearer's nose. The side wall may have a greater depth in the nasal region than in the chin or cheek regions. The depth of the side wall in the chin and cheek regions may be substantially the same. The cheek regions may not comprise a side wall.

The depth of the side wall may gradually decrease between the nasal region and the cheek region, such that the region of the sidewall having the greatest depth is in the nasal region and a region of the sidewall having the smallest depth is in the cheek region. The depth of the sidewall may be substantially constant between the chin region and the cheek regions. Where present, one or more seal supporting members may extend outwardly from the side wall, eg a seal supporting member may extend outwardly from a central point in the chin region.

A first region of the sidewall may extend between the nasal region and a cheek region. The first region of the sidewall may be generally arcuate in shape and may extend rearwardly from the rim of the support frame (ie towards the wearer's face, in use). At least a portion of the first region of the sidewall may extend rearwardly from the rim of the support frame at an obtuse angle relative to the front face of the mask. The portion of the first region adjacent the nasal region may extend outwardly at an obtuse angle relative to the front face of the face mask. At least a portion of the first region of the sidewall may extend rearwardly from the rim of the support frame at a right angle relative to the front face of the mask, that is, in a direction perpendicular to the front face of the face mask. The portion of the first region adjacent the cheek region may extend rearwardly from the rim at a right angle relative to the front face of the mask.

A second region of the sidewall may extend between the chin region and a cheek region. The second region of the sidewall may be generally arcuate in shape.

Where the face mask is a filter mask, the mask body may be defined by the support frame and the filter. The sealing member may extend about the periphery of the support frame, eg about the periphery of the rim. The shape of at least a portion of the support frame and of the filter may correspond to the shape of the proximal edge of the sealing member. The support frame and the filter may extend laterally across the wearer's cheeks. The portions of the mask body which extends across the wearer's cheeks may form a shallower cavity between the mask and the wearer's face than the portion of the mask which extends over the wearer's nose and mouth.

The extension of the mask over the cheeks increases the surface area of the sheet of filtering material, thus increasing the volume of air which can pass through the filter. At the same time, the shallower cavity in this region of the mask reduces the dead space within the mask.

Embodiments in which the cheek portions extend laterally across the wearer's cheeks may be advantageous in therapy masks, including but not limited to nebuliser masks, sleep apnoea masks, CPAP masks, oxygen delivery masks, NIV masks and aerosol masks. The breadth of the mask in the cheek regions provides a more stable fit on the wearer's face, making it less likely to move or become dislodged during use.

Masks according to this embodiment of the invention may be particularly advantageous in situations where the user moves considerably, such as for the treatment of sleep apnoea, and for use in CPAP and non-invasive ventilation. The wearer in such situations is typically a patient who may move and roll, and the low profile of the mask and increased stability provided by the lateral extension of the cheek portions allows the wearer to move more easily, even while asleep, without dislodging the mask. In addition, for elderly patients or other patient groups who may have lost some teeth, the lateral extension of the seal over the cheeks enables a good seal to be obtained with less force in this area. The seal may be overmoulded onto the mask body, or may be manufactured as a separate component, which is attached to the mask body. Appropriate materials for the production of the mask body and seal, and their suitability for overmouding, would be readily apparent to the person skilled in the relevant technical field (eg materials science). The mask may not include a filter. The mask may include a filter, which may provide controlled leakage of air at a particular pressure. Typically, controlled leaks in masks can cause noise which is irritating to the wearer, and the use of a filter or other permeable material, eg a textile material, to provide a controlled leak would reduce or prevent noise.

As the effectiveness of the mask increases with improvement in fit (and reduction in leaks), it is useful to have a mask that may accommodate faces of different sizes, and which remains effective even where the wearer is moving and carrying out an active role, such as in a filter mask for use by a healthcare professional.

According to a further aspect of the invention, there is provided a filter mask comprising a support frame and a sheet of filtering material, the support frame and the sheet of filtering material defining a mask body adapted to provide a cavity in use about the mouth and nose of a wearer, the filter mask includes a sealing member depending from at least a portion of a peripheral edge of the mask body, the sealing member comprising both an inwardly and outwardly depending lip portion relative to the peripheral edge of the mask body.

The sealing member may extend around the entire peripheral edge of the mask body. The portion of the peripheral edge of the support frame about which the inwardly and outwardly depending lip portions extend may comprise a chin portion. The inwardly depending lip portion may extend around a majority of the peripheral edge of the support frame. The inwardly depending lip portion may extend around only a part of the peripheral edge of the support frame. The portion of the peripheral edge of the support frame about which the inwardly depending lip portions extend may comprise a chin portion, and/or may comprise a nasal portion. The portion of the peripheral edge of the support frame about which the inwardly depending lip portions extend may not comprise a cheek portion, to facilitate removal of the seal from the mould. The portion of the peripheral edge of the support frame about which the inwardly depending lip portions extend may comprise a cheek portion, to provide an improved seal. The outwardly depending lip portion may extend around a majority of the peripheral edge of the support frame. The outwardly depending lip portion may extend around only a part of the peripheral edge of the support frame, eg the chin and cheek regions. The nasal portion of the sealing member may not comprise an outwardly depending lip portion.

It has been found that the utility of the above described features extends beyond their use in filter masks, and that a seal having the above described features may be applied to any face mask in which an effective seal is required.

Thus, there is provided a face mask comprising a sealing member depending from at least a portion of a peripheral edge of the mask body, the sealing member comprising a chin region, a nasal region, and cheek regions intermediate the chin and nasal regions, and the sealing member comprising both an inwardly and outwardly depending lip portion relative to the peripheral edge of the mask body, wherein the inwardly depending lip portion extends about the chin and nasal regions of the sealing member, and the outwardly depending lip portion extends about the chin and cheek regions of the sealing member.

It is important to form a good seal against the face, whilst not driving up the cost and complexity of manufacture. It has been found that, while an inwardly depending lip portion provides a good seal against the face, it can hinder manufacture by increasing the difficulty of removing the seal from the mould. By providing an inwardly depending lip portion in the chin and nasal regions, which have the greatest variation in shape/size, the seal is improved. The cheeks are typically flatter and formed of softer tissue and forming a good seal against the cheeks is therefore easier. Thus, only an outwardly depending lip portion in these areas is sufficient to provide a good seal, whilst facilitating manufacture.

The inwardly depending lip portion may be generally planar in form. The inwardly depending lip portion may take the form of an upstanding wall which follows the contour of the peripheral edge of the body. The inwardly depending lip portion may include discontinuities therein that facilitate deformation of the inwardly depending lip portion.

At least a portion of the outwardly depending lip portion may have a contact surface that is concave in shape. The outwardly depending lip portion may be shaped so as to provide a sill or shelf formation beneath the wearer's mandible. The outwardly depending lip portion may take the form of an upstanding wall which follows the contour of the peripheral edge of the body. At least a portion of the outwardly depending lip portion may be planar, and about the wearer's face, eg in the chin region and/or in the cheek region. A proximal portion of the outwardly depending lip portion may engage the chin of the wearer, while a distal portion curves and extends under the chin of the wearer. The outwardly depending lip formation may be shaped in the form of a chin cup. The chin cup may, in use, accommodate at least a portion of the chin of the wearer. The outwardly depending lip portion may be shaped so as to provide both a front lip portion and also an underside lip portion.

The inwardly depending lip portion may contact a lower portion of the wearer's face, eg the wearer's chin, to form a seal. Where a user has a larger face height, the outwardly depending lip portion may pass closely beneath the mandible of a wearer, providing an additional seal. For all wearers, the outwardly depending lip portion may provide an alignment guide for placing the mask on the face, and help to keep the mask in the correct position on the face despite movement of the jaw during use.

The periphery of the support frame in the vicinity of the inner and outer lip portions, in the chin region, may be shaped from a central point or region of the mask by a first radius. The inwardly depending lip portion typically has an inner edge of radius which is smaller than the first radius. The outwardly depending lip portion typically has an outer edge of radius which is larger than the first radius.

The nasal portion of the sealing member may comprise an inwardly depending lip portion, which may present a convex surface for contact with a wearer's face. The nasal portion of the sealing member may include reinforcement formations, eg reinforcement formations on each side of the nose, or reinforcement formations about the entirety of the nasal region. It has been found that reinforcement formations in the nasal region provide stability to the mask, preventing unwanted movement and/or collapse of the seal while maintaining the lateral flexibility required to accommodate different face sizes, contributing to the formation of a reliable seal. This reduces unwanted deformation in this area, or unwanted folding of the seal, improving the fit and making correct fitting of the mask easier.

Such a filter mask may comprise cheek portions which extend laterally across the wearer's cheeks as in the embodiment previously described, and comprising any of the features of that embodiment, the cheek portions extending laterally between the upper edges of the malar bones and the lower edge of the mandible of the wearer. This increases the stability of the mask on the face.

The face mask according to the invention may include means for securing the mask to the wearer, in use. Such means may include one or more cords or straps, for example an elasticated cord or strap, that is fitted around the wearer's head to urge the filter mask against the face of the wearer.

The one or more cords or straps may be formed as a separate component or components, and subsequently be attached to the filter mask either by insertion through a hole or slot and knotting or sealing, or by bonding the cord or strap to the mask. The cord(s) or strap(s) may be bonded to the mask by an adhesive or chemical bond, or by overmoulding. The cord(s) or strap(s) may be worn over the ears of the wearer, or around the back of the wearer's head. This may help prevent a mask from slipping down and/or rising up a wearer's face in use.

Two cords or straps may be provided, to pass around each of the wearer's ears, or to extend around the back of the head, one cord passing above and one below the wearer's ears. A single cord may be provided, which extends about the back of the wearer's head.

Alternatively, a single cord may pass twice across the back of the wearer's head. A first side of the support frame may be located, in use, adjacent to one of the wearer's ears while a second side of the support frame is located, in use adjacent to the other of the wearer's ears. The first and second sides of the support frame may be located in the regions of the support frame which, in use, are closest to the wearer's ears.

The elasticated cord or strap may comprise means for indicating that a desired fit has been achieved. The elasticated cord or strap may comprise indicia which indicate to the user that their desired fit has been achieved. The elasticated cord or strap may comprise a size guide. Once a mask according to the invention has been worn and adjusted to provide the correct fit eg using a fit test, the user may use the indicia to adjust any further mask to the correct size, without the need for a further fit test.

Where the mask is a filter mask, the mask may comprise an aperture in the filter. The aperture may provide a conduit through the filter, or may include or be adapted to receive a further device, such as an exhalation valve. The aperture may bypass the filter material. The aperture in the filter material may be mounted and/or sealed to an aperture in the support frame or to a device mounted to the support frame. Furthermore, the polymeric material that is injected into the mould to form the sealing member may be brought into engagement with the filter and the aperture in the support frame or to the device mounted to the support frame, during injection moulding of the sealing member, in a manner that seals the filter about the aperture or device.

The mask may comprise one or more vents or valves, for example an exhalation valve. An exhalation valve may enable exhaled air to escape from the filter mask without passing through the filter, therefore reducing the heat and humidity retained by the filter, during use. However, the inclusion of an exhalation valve may not be desirable where the filter mask is needed to protect others from pathogens exhaled by the wearer, eg in a healthcare environment. The vent or valve may be located in the shield, or in the rim of the support frame. The valve may be inserted into an aperture in the shield or rim, or may form part of the shield or rim. Where the valve is inserted into or forms part of the shield, it may also extend through the filter material, and the overmoulding step which forms the sealing member may be used to additionally provide a seal between the valve and the filter material and/or between an aperture in the support frame for receiving the valve and the filter material. Where the valve is inserted into or forms part of the rim, a portion of the rim may be shaped to accommodate the valve, eg in the chin region.

Where a valve is included, the injection moulding step for forming the sealing member may also form the valve member. The sealing member and the valve member may therefore be integrally formed. After moulding of the sealing member/exhalation valve, the valve may be inserted either through openings in the filter and shield, or through an opening in the rim. Tension between the valve and the surrounding material (shield, filter or rim) biases it closed. This permits exhaled air to flow from the interior of the mask to the exterior, but prevents the flow of air in the opposite direction.

The support frame, eg the shield, and/or filter may be formed with an aperture to which either a releasable cover or an exhalation valve may be fitted. The overmoulding step which forms the sealing member may be used to additionally provide a seal between the aperture in the support frame for receiving the valve or cover and the filter material. For example, the releasable cover may be secured over the aperture using a snap-fit or screw fit connection, the cover being removed and replaced with an exhalation valve where required. This enables a single mask to be produced which may be used either with or without an exhalation valve. The size of the aperture will be an appropriate size for the attachment of an exhalation valve, and may be 10-50 mm in diameter, or 15-40 mm in diameter, or 15-30 mm in diameter. Typically, the diameter of the aperture will be approximately 20 mm.

The filter mask may further comprise a spigot, which may be used to connect the mask to a tube for introducing gases to, or removing gases from, the mask and/or the wearer. Thus, the filter mask may be used as an oxygen delivery mask or respirator, with the additional protection that inhaled and exhaled air (other than the oxygen being delivered) must pass through the filter. In the same way as with the exhalation valve, the spigot may be releasably attached to an aperture in the shield and, optionally, the filter. The aperture may be sized to accommodate a spigot having a diameter of from 10-50 mm in diameter, or 15-40 mm in diameter, or 15-30 mm in diameter. Alternatively, the aperture may be sized to accommodate a spigot having a diameter of from 2-10 mm in diameter, or 2-8 mm in diameter, or 4-8 mm in diameter. The spigot may alternatively be integrally formed with the shield, or with another part of the support frame.

The filter mask may comprise a filter and a gas inlet (eg a spigot), and be suitable for use as a high concentration mask, as an oxygen mask, as a CPAP mask or as a high flow mask. Conventional masks for use in the delivery of high concentrations of oxygen require a reservoir bag to be attached to the inlet, to provide a reservoir of oxygen to be drawn on in the event that the patient breathes in excess of what the oxygen supply can meet. It is believed that the larger internal volume of the mask herein described when compared to conventional masks, particularly where the mask extends laterally across the wearer's cheeks, may remove the need for an additional reservoir bag. In addition, the need to control infection and the spread of contaminants means that the inclusion of a filter is highly desirable, protecting both a patient and those providing their treatment.

The filter mask may comprise a gas inlet (eg a spigot) which extends through or is integrally formed with the support frame or mask body, and a filter. The gas inlet and the filter may be located in different parts of the support frame or mask body. The gas inlet and the filter may be located in first and second regions of the support frame or mask body. The first and second regions may be located on different sides of the wearer's nose and mouth, in use. The filter may be located in a first cheek region of the support frame, and the gas inlet located in a second cheek region of the support frame, such that oxygen flows from the gas inlet to the filter, washing over the wearer's mouth and nose, thus providing a continuous flow of oxygen and helping to draw away exhaled gases.

The support frame or mask body may comprise a housing having a first aperture into which a filter may be received, and a second aperture thorough which a gas inlet may be inserted. Alternatively, the gas inlet may be at least partially integrally formed within the support frame or mask body. It is believed that the reservoir of air held within the filter mask may remove the need for an additional reservoir bag. In addition, the need for an anti-asphyxiation valve is reduced or removed as, in the event of oxygen supply failure, the wearer may inhale ambient air through the filter. The lack of need for a reservoir bag and anti-asphyxiation valve reduces both the complexity and the cost of manufacture.

Alternatively, the filter mask may not comprise a spigot. Such filter masks may be unsuitable for use as an oxygen delivery mask or a respiratory mask.

The filtration mask may additionally or alternatively comprise an access port. For example, the filtration mask may comprise an access port for an endoscope, a nasal cannula, or a high flow nasal cannula, such that an endoscopy may be carried out or cannula worn while still providing the patient and medical practitioner with the protection offered by a filtration mask. Masks having cheek portions that extend laterally over the cheeks may be particularly advantageous for use as endoscopy masks, due to the additional stability provided by the lateral extensions which reduce the need for additional head straps to stabilise the endoscopy mask. Medicaments or oxygen may be delivered in through a high flow nasal cannula, and it is a feature of this type of treatment that aerosolised particles may be generated and released to the environment. Depending on the nature of the patient's illness, such aerosolised particles may be contaminated and pose a risk to those in the vicinity. Wearing a filter mask which allows for access to the airway to deliver a high flow nasal cannula but which contains any generated aerosol thus provides a safer environment for clinicians and other personnel in the vicinity. The access port may comprise an aperture in the filter mask. The aperture may comprise a pair of apertures in the support frame, eg shield, and sheet of filtering material that are in registration with each other, such that a cannula can pass through both apertures. The overmoulding step which forms the sealing member may be used to additionally provide a seal between the aperture in the support frame and the filter material. The apertures in the support frame, shield and/or filtering material may be provided with removable covers, such that they may be covered when not in use.

In use, the wearer of the face mask may exhale expiratory gases into the interior cavity. Thus, A face mask according to the invention, which may be a filter mask, may additionally comprise a sensor to detect the CO₂ concentration in exhaled air, such as that described in GB2019236.5 (which is incorporated herein by reference).

In this embodiment the mask body and/or the support frame may be formed of an IR-transparent material. The sensor may be mountable relative to the sensor portion of the face mask. The sensor may have a transmitter of electromagnetic radiation and a receiver of electromagnetic radiation, wherein an enclosing wall of the mask body has a sensor portion including first and second sensor windows, a portion of the interior cavity defined by the enclosing wall being defined between the first and second sensor windows, and the sensor being mounted relative to the sensor portion of the respiratory mask, such that electromagnetic radiation from the transmitter is transmitted, in use, through the first sensor window of the sensor portion of the enclosing wall, through the portion of the interior cavity defined between the first and second sensor windows, through the second sensor window, to the receiver. A face mask having a sensor as described may be advantageous in that the sensor portion being formed in the enclosing wall of the mask body allows the sensor to monitor the patient's expiratory gases before they leave the mask body, reducing the delay between the gases being exhaled and the measurements being taken. In contrast, using conventional respiratory masks that are connected to a sensor via an exhalation tube or a sampling tube, expiratory gases exhaled by the wearer have to travel from the respiratory mask to the sensor, eg a respiratory monitoring unit, before they can be monitored. Each of the first and second sensor windows may have an interior surface and an exterior surface. The sensor may be mounted relative to the sensor portion of the respiratory mask with the transmitter disposed adjacent to the exterior surface of the first sensor window and the receiver disposed adjacent to the exterior surface of the second sensor window.”

“The electromagnetic radiation transmitted by the transmitter and received by the receiver may be infrared radiation. Infrared radiation may refer to radiation having a wavelength between approximately 700 nm and approximately 1 mm.”

“The first and second sensor windows may be transmissive of the electromagnetic radiation transmitted by the transmitter. For example, the first and second sensor windows may be transmissive of infrared radiation, ie radiation having a wavelength between approximately 700 nm and approximately 1 mm. The first and second sensor windows may also be transmissive to other wavelengths of light, such as visible light. The first and second sensor windows may be more transmissive of the electromagnetic radiation transmitted by the transmitter than the remainder of any of, or any combination of, the mask body, the enclosing wall of the mask body, and the sensor portion.”

“The first and second sensor windows may be transmissive to at least 50%, 60%, 70%, 80%, 90% or 100% of the electromagnetic radiation transmitted by the transmitter. To obtain an accurate and/or viable reading, the sensor may require that a minimum amount of electromagnetic radiation, or a minimum proportion of the electromagnetic radiation transmitted by the transmitter, is received by the receiver. Hence, the first and second sensor windows may be transmissive of the electromagnetic radiation transmitted by the transmitter such that at least 20%, 30%, 40%, 50%, 60%, 70%, 80% or 90% of the electromagnetic radiation is received at the receiver after passing through the portion of the interior cavity defined between the first and second sensor windows.”

“The first and second sensor windows may be of a reduced thickness relative to the remainder of any of, or any combination of, the mask body, the enclosing wall of the mask body, and the sensor portion. The first and second sensor windows may have a thickness of up to 0.5 mm, up to 0.4 mm, up to 0.3 mm, up to 0.2 mm, up to 0.1 mm, or up to 0.05 mm. The thickness of the first and second sensor windows may refer to the distance between the interior surface and the exterior surface of each sensor window.”

“The first and second sensor windows, eg the interior surface of the first and second sensor windows, may be spaced apart by a distance of between 2 mm and or between 5 mm and 30 mm, ie the portion of the interior cavity defined between the first and second sensor windows may have a thickness of between 2 mm and 50 mm, or between 5 mm and 30 mm.

Methods for the manufacture of sealing members are known in the art, and any suitable method may be used in combination with the present invention. In particular, the sealing member may be at least partially manufactured using a gas-assisted injection moulding method as described in WO2021/037768 and GB2013108.2 (which are hereby incorporated by reference). These documents describe the use of gas-assisted injection moulding in the manufacture of a sealing member for a respiratory device. Filtration masks for personal protection require a very effective seal, and it has surprisingly been found that sealing members may be produced by this method which provide the highly effective seal required in a filtration mask.

According to a further aspect of the invention, there is provided a method of manufacturing a sealing member for use in a face mask, the method comprising the steps of:

(a) providing a mould having a cavity, a polymer injection port and a gas inlet port;
(b) injecting a polymer through the polymer injection port into the cavity of the mould; and
(c) introducing gas through the gas inlet port into the cavity of the mould,
   thereby forming a sealing member of the face mask, wherein at least a portion of the sealing member of the face mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user.

The face mask according to this aspect of the invention may be a respiratory mask. The face mask according to this aspect of the invention may be a filter mask.

It has been found that, when used to manufacture at least a portion of a sealing member for a face mask, in particular for a respiratory or filter mask, the external surface of the resiliently deformable enclosing wall can be shaped to provide an anatomical fit with a wearer or wearer's face, providing the sealing member with an effective seal, whilst the internal chamber bounded by the enclosing wall also enables the sealing member to be urged against the wearer, eg against the wearer's face, in the event that the seal needs to be improved. An effective seal is particularly important in a filtration mask as, if there are leaks, air will circumvent the filter and the mask will lose its efficacy.

Only a portion of the sealing member may be produced according to the above method. It is particularly advantageous for the nasal region of the sealing member of a face mask, eg a respiratory mask or a filtration mask, to be produced using the above-described method. Thus, a sealing member may be provided in which only the nasal region is produced according to the above-described method. The contours of the nose make it difficult to form an effective seal in this region. It has been found that a sealing member wherein the nasal region is produced by gas-assisted injection moulding as described forms a particularly effective seal in the nasal region, as the seal is less susceptible to buckling, folding and/or otherwise deforming, which may create leaks in the seal.

When manufacturing sealing members using gas-assisted injection moulding, it was found that it was advantageous to inject at least a minimum volume of polymer to prevent the injected gas from blowing through the exterior surface of the polymer upon gas injection, which would create a hole (or at least a deformed or weakened portion) in the external surface of the resultant sealing member. Furthermore, the injection of a greater volume of the polymer was also found to result in a more uniform enclosing wall and internal chamber in the resultant sealing member.

However, it was found that the volume of polymer required to provide these advantages may result in an enclosing wall having a greater thickness than desired, which increased the resilience of the external surface of the sealing member, and when used in respiratory interface devices, may make it more difficult to urge against a wearer's face to create a seal, meaning a softer material may have to be used to form the sealing member, which would be undesirable.

It was found that the introduction of a blowing agent, mixed with the polymer, and the expansion of the blowing agent in the cavity of the mould, may increase the volume of the cavity that is taken up by the polymer body after injection into the cavity of the mould, without increasing the thickness of the enclosing wall in the resultant sealing member. In particular, because the blowing agent expands within the polymer, the volume of the mix of the polymer and the blowing agent may be greater than the volume of the polymer if it were to be injected alone. The surface area of the polymer may also be increased relative to a polymer injected without a blowing agent. This may allow less polymer to be injected and a reduced enclosing wall thickness in the resultant sealing member, whilst still achieving the minimum volume of polymer body required in the cavity to prevent blow-through when the gas is introduced and provide a more uniform enclosing wall and internal chamber in the resultant sealing member.

Without wishing to be bound by theory, it is thought that the increased volume of the cavity that is taken up by the polymer body may cause the polymer body to contact the walls of the cavity. Since the walls of the cavity are the coolest part of the cavity, the polymer and blowing agent mix that is in contact with the walls may cool first, and a solidified (or at least more viscous) skin layer may be formed at the walls of the cavity. It is thought that this may lead to a hardened or more viscous skin layer at the walls of the cavity, which may encompass a less viscous core of the polymer body that extends along a central longitudinal axis of the cavity of the mould. This skin layer and less viscous core is thought to create a path of least resistance for the gas—along a central longitudinal axis of the cavity of the mould. It is thought that, when introduced, the gas may push the core of the polymer body, or a substantial part thereof, through the cavity, with the skin layer remaining at the walls of the cavity, thereby forming an internal chamber that is at least partially bounded by an enclosing wall of polymer.

It is thought that the creation of a path of least resistance along the central longitudinal axis of the cavity of the mould may be enhanced by the formation of gas pockets resulting from the blowing agent. Whilst the polymer and blowing agent mix retains sufficient heat, the blowing agent may expand to form gas pockets in the polymer and blowing agent mix within the cavity. Since the walls of the cavity are the coolest part of the cavity, the polymer and blowing agent mix in contact with the walls may cool first and thus expansion of the blowing agent at the walls of the cavity may slow and/or stop first. This may lead to a difference in the size of gas pockets formed in the polymer and blowing agent mix within the cavity, the size of the gas pockets increasing with distance from the walls of the cavity. This difference in size is thought to enhance the creation of a path of least resistance along a central longitudinal axis of the cavity of the mould.

Hence, the addition of a blowing agent may enable greater control and predictability over the final form of the sealing member, without increasing the thickness of the enclosing wall, for example.

There is thus provided a method of manufacturing at least a portion of a sealing member for use in a face mask, the method comprising the steps of:
(a) providing a mould having a cavity, a polymer injection port and a gas inlet port;
(b) injecting a polymer and a blowing agent through the polymer injection port into the cavity of the mould; and
(c) introducing gas through the gas inlet port into the cavity of the mould, to form a sealing member,
   wherein at least a portion of the sealing member comprises an internal chamber at least partially bounded by a resiliently deformable enclosing wall formed of the polymer, the enclosing wall including an external surface, the external surface having a form that is determined by the cavity of the mould.

The sealing member produced according to this method may be for use in a respiratory mask. The sealing member produced according to this method may be for use in a filter mask.

Only a portion of the sealing member may comprise an internal chamber at least partially bounded by a resiliently deformable enclosing wall formed of the polymer, the enclosing wall including an external surface, the external surface having a form that is determined by the cavity of the mould. Only the nasal portion of the sealing member may comprise an internal chamber at least partially bounded by a resiliently deformable enclosing wall formed of the polymer, the enclosing wall including an external surface, the external surface having a form that is determined by the cavity of the mould.

The blowing agent may be a substance that, when mixed with the polymer, expands to produce gas pockets within the polymer, for example via a foaming process, and the polymer undergoes a period of hardening, or a phase transition, following formation of the gas pockets. The blowing agent may therefore cause the polymer to adopt a cellular structure.

The blowing agent may be a physical blowing agent. It is believed that the provision of a physical blowing agent is advantageous because, at a given temperature and pressure, a physical blowing agent has a limit to its expansion, which enables a greater predictability of the physical blowing agent's behaviour, and hence a greater control over the expansion of the blowing agent and the resultant sealing member.

The physical blowing agent may be caused to expand by one of, or a combination of, the application of heat and a reduction in pressure on injection of the polymer and blowing agent mix into the cavity of the mould. The polymer and physical blowing agent mix may be heated before injection into the cavity of the mould and/or the cavity of the mould may be heated. The reduction in pressure may be achieved by the cavity of the mould only being partially charged by the polymer and physical blowing agent mix in the injection moulding step.

Where gas is produced by the physical blowing agent, it may be by a physical process, eg by a phase change, such as the vaporisation of a liquid. The production of gas by the physical blowing agent may involve no chemical reactions. In contrast, the production of gas in a chemical blowing agent is by means of a chemical reaction. The gas produced by the physical blowing agent may expand, and hence its volume increase, by a physical process, eg by an increase in temperature and/or a reduction in pressure.

It is believed that a physical blowing agent that acts in this way is advantageous because it expands under a wider range of conditions, and is therefore more adaptable. For example, although there is an optimum working temperature at which the aforementioned physical blowing agent will expand most efficiently, it is believed that they still expand controllably over a wide temperature range.

The physical blowing agent may have a multi-layered structure. For example, the physical blowing agent may have a core-shell structure. The shell layer may surround or encapsulate the core layer. The core-shell structure may be substantially spherical. The shell may be formed of a solid material, such as a polymer. The polymer may be a thermoplastic, such as an acrylonitrile polymer or a nitrile, eg polyacrylonitrile or acrylonitrile. The core layer may comprise the gas or gas-producing material of the physical blowing agent, which may be a liquid. The gas or liquid may be a hydrocarbon, such as an octane isomer, eg trimethylpentane, or a pentane isomer, eg pentane or isopentane, or any combination thereof. In the trade, blowing agents having this structure may be referred to as microspheres. The core layer may produce gas and/or expand within the shell layer in response to the application of heat from a source external of the blowing agent. The polymer (and blowing agent) may heat to a temperature of 50-300 degrees Celsius, 75-250 degrees Celsius, or 80-235 degrees Celsius. The volume of the core layer and/or the microsphere may increase in response to the application of heat and/or a reduction in pressure from a source external of the blowing agent. The diameter of the core layer and/or the microsphere may increase in response to the application of heat and/or a reduction in pressure from a source external of the blowing agent. The thickness of the shell layer may decrease in response to the application of heat and/or a reduction in pressure from a source external of the blowing agent. In a particularly preferred embodiment, the physical blowing agent may be a liquid that can be vaporised to produce gas, the liquid being surrounded or encapsulated by a solid shell layer.

Prior to the expansion within the polymer, the diameter of the microsphere may be in the range of 5-20 μm, 8-15 μm, or 10-12 μm. Following expansion within the polymer, the diameter of the microsphere may be in the range of 10-1000 μm, 25-750 μm, or 50-500 μm, 10-200 μm, 20-160 μm, or 30-140 μm.

Prior to the expansion within the polymer, the thickness of the shell layer may be in the range of 1-2.5 μm, 1.25-2.25 μm, or 1.5-2 μm. Following expansion within the polymer, the thickness of the shell layer may be in the range of 0.01-0.25 μm, 0.05-0.2 μm, or 0.1-0.15 μm. Following expansion within the polymer, the thickness of the shell layer may decrease to 1-20%, 2.5-15%, or 5-10% of the thickness of the shell layer prior to the expansion within the polymer.

The shape of the external surface may be determined by the cavity of the mould. The dimensions of the external surface may be determined by the mould, such as the radius and/or the diameter of the external surface. The volume of the sealing member may be determined by the cavity of the mould. The final form of the material formed from the polymer and the blowing agent may be determined by the cavity of the mould. The shape and/or dimensions of the external surface may match, or correspond to, the shape and/or dimensions of the cavity of the mould.

The volume of the sealing member may match, or correspond to, the volume of the cavity of the mould.

The injection of gas through the gas inlet port into the cavity of the mould may be maintained until the sealing member adopts its final form. However, the pressure of the injected gas may change after formation of the internal chamber. For example, the pressure may be reduced, provided there is enough pressure to prevent the sealing member from separating from the walls of the cavity and collapsing inwardly into the cavity of the mould. Once the blowing agent has completed its expansion in the polymer-blowing agent mix, the continued injection of gas may cause the volume of the mix of the polymer and the blowing agent to be reduced.

The blowing agent may be mixed with the polymer prior to injection through the polymer injection port into the cavity of the mould, and the gas may be first introduced through the gas inlet port into the cavity of the mould after the cavity of the mould is at least partially charged by the mixed polymer and blowing agent. This feature may facilitate formation of a uniform internal chamber. During mixing of the blowing agent with the polymer, the blowing agent may be uniformly distributed throughout the polymer, for example using a volumetric dosing unit. This ensures that the blowing agent expands uniformly and predictably throughout the cavity of the mould.

According to a further aspect of the invention, there is provided a sealing member for a face mask, eg for a respiratory mask or a filter mask, at least partially manufactured by the method described above. According to a further aspect of the invention, there is provided a sealing member for a face mask, eg for a respiratory mask or a filter mask, wherein the nasal region is manufactured by the method described above. According to a further aspect of the invention, there is provided a sealing member for a face mask, eg for a respiratory mask or a filter mask, wherein only the nasal region is manufactured by the method described above.

According to a further aspect of the invention, there is provided a sealing member for a face mask, eg for a respiratory mask or a filter mask, at least a portion of the sealing member comprising an internal chamber at least partially bounded by a resiliently deformable enclosing wall, the enclosing wall including an external surface, the external surface having a form that is determined by the cavity of the mould.

The enclosing wall may have a plurality of gas pockets formed therein. The external surface may comprise a face-contacting surface, and the face-contacting surface may provide an anatomical fit with a patient.

The face-contacting surface of the sealing member may be formed with a pre-determined anatomical shape. The face-contacting surface of the sealing member may have the form of a closed loop, for example extending around the mask body.

The sealing member may have the form of a loop, with the internal chamber of the sealing member being continuous and extending around at least a majority of the loop.

Alternatively, only a portion of the sealing member may comprise an internal chamber at least partially bounded by a resiliently deformable enclosing wall, the enclosing wall including an external surface, the external surface having a form that is determined by the cavity of the mould. Only the nasal portion of the sealing member may comprise an internal chamber at least partially bounded by a resiliently deformable enclosing wall, the enclosing wall including an external surface, the external surface having a form that is determined by the cavity of the mould, while the cheek regions and chin region of the sealing member comprise an outwardly and/or inwardly depending lip portion, the entire sealing member depending from at least a portion of a peripheral edge of the mask body. The sealing member in the chin and cheek regions may have thickness of not more than 2 mm, or not more than 1.5 mm. The nose has a complex topography and it is therefore difficult to create an effective seal against it. It has been found that the use of gas-assisted injection moulding to produce the seal only in the nasal region results in an enhanced seal in this area while keeping manufacturing costs down.

The gas in the gas pockets may be gas produced by the use of a blowing agent during manufacture of the sealing member, for example by the methods of manufacture described above, or may be ambient air that has filled the pockets after manufacture of the sealing member. The gas pockets may be akin to bubbles, a cellular structure, or a matrix of holes in their appearance. The majority of gas pockets formed in the enclosing wall may have a diameter in the range of μm, 25-750 μm, or 50-500 μm, 10-200 μm, 20-160 μm, or 40-120 μm.

The sealing member may include an aperture in fluid communication with the internal chamber of the sealing member and with ambient air, such that ambient air may enter and exit the internal chamber during use. Where multiple internal chambers are provided, each internal chamber may be provided with an aperture in the enclosing wall of the sealing member. The aperture, or apertures, may be provided in the enclosing wall of the sealing member. If the seal needs to be improved, in use, for example in therapy, the user may urge the sealing member against the wearer's face, eg by applying pressure on the respiratory device towards the wearer's face. The sealing member and the internal chamber would be compressed, causing air to exit the internal chamber, but the resilience of the sealing member may be sufficient for the internal chamber not to fully collapse, ie there remains a separation between opposing internal surfaces of the enclosing wall, and to return to its original shape once the pressure has been removed. Even without additional pressure, or where the pressure of the mask against the face is retained by one or more straps extending about the wearer's ears and/or head, this seal has been found to provide an effective seal which withstands the pressures and movements of normal use. In particular, movement of the mask against the face may cause sideways pressure on the seal, but the ability of the enclosed sealing member to roll against the face rather than buckling or distorting helps to prevent leaks.

This method of manufacturing a sealing member may be used in the manufacture of a sealing member for use in the filtration mask of the first aspect of the invention. According to a further aspect of the invention there is thus provided a method of manufacturing a filter mask, the filter mask comprising a support frame, a filter and a sealing member, wherein the method comprises:
a) locating the filter and the support frame in a mould; and
b) forming the sealing member by:
   i) providing a mould having a cavity, a polymer injection port and a gas inlet port;
   ii) injecting a polymer through the polymer injection port into the cavity of the mould; and
   iii) introducing gas through the gas inlet port into the cavity of the mould, thereby forming a sealing member of the filter mask, wherein at least part of the sealing member of the filter mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user;
      and wherein the sealing member is brought into engagement with the filter and the support frame, during injection moulding of the sealing member, in a manner that fixes the sealing member to the support frame and the sheet of filtering material.

A blowing agent may be injected with the polymer in step ii).

Only a portion of the sealing member may comprise an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user. Only the nasal portion of the sealing member may comprise an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user, while the cheek regions and chin region of the sealing member may comprise an outwardly and/or inwardly depending lip portion, the entire sealing member depending from at least a portion of a peripheral edge of the mask body.

The enclosing wall may comprise a plurality of gas pockets formed therein, as previously described. The face-contacting surface may provide an anatomical fit with a patient.

The face-contacting surface may provide an anatomical fit with a patient before any deformation of the sealing member in use, ie the face-contacting surface may be anatomically shaped. In order to provide an anatomical fit, the face-contacting surface may have a leading portion, ie a portion that contacts a surface of the user before any deformation of the sealing member, that is anatomically shaped. The anatomical shape may be determined at least in the direction of engagement of the sealing member with a surface of the user, such that the position of the leading portion of the face-contacting surface varies in this direction, eg at different positions along the face-contacting surface. The leading portion of the face-contacting surface may have the form of a closed loop, for example extending around a mask body for a respiratory mask. The leading portion and/or a central line on the leading portion of the face-contacting surface may have a varying position relative to a reference surface, such as a reference plane or a reference cylindrical surface, where the reference surface may be arranged perpendicularly to the direction of engagement of the sealing member with the surface of the user, or the direction of global pressure applied by the sealing member to the surface of the user. The leading portion and/or a central line on the leading portion of the face-contacting surface may have a position relative to a reference surface that varies non-linearly.

According to a further aspect of the invention, there is provided a filter mask manufactured according to the first aspect of the invention, wherein at least a portion of the sealing member comprises an internal chamber at least partially bounded by a resiliently deformable enclosing wall, the enclosing wall including an external surface, the external surface having a form that is determined by the cavity of the mould. According to a further aspect of the invention, there is provided a filter mask comprising a support frame, a filter and a sealing member, wherein the sealing member is fixed to the support frame and the filter, and wherein at least a portion of the sealing member comprises an internal chamber at least partially bounded by a resiliently deformable enclosing wall, the enclosing wall including an external surface, the external surface having a form that is determined by the cavity of the mould. Only the nasal portion of the sealing member may comprise an internal chamber at least partially bounded by a resiliently deformable enclosing wall, the enclosing wall including an external surface, the external surface having a form that is determined by the cavity of the mould.

It will be understood that any features described in relation to one aspect of the invention may be applied to any other aspect of the invention. Thus, any structural features described in relation to a mask having integral filter, such as a shield, cord or elastic strap, exhalation valve and/or spigot, may be applied to the mask having a sealing member at least partially produced by gas-assisted injection moulding.

Embodiments of the invention are now illustrated, by way of example only, with reference to the following drawings:

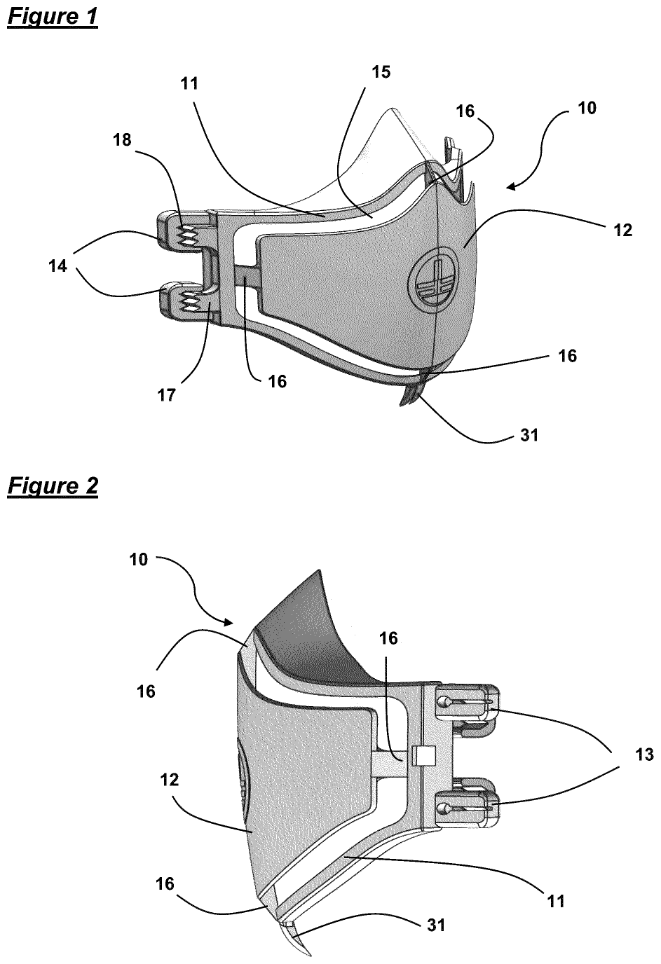

FIG. 1 is a perspective view of a support frame of a first embodiment of a filtration mask according to the invention;

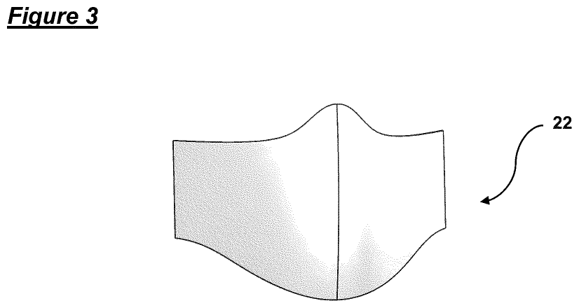

FIG. 2 is a side view of the support frame of FIG. 2;

FIG. 3 is a perspective view of a filter of the first embodiment of a filter mask according to the invention;

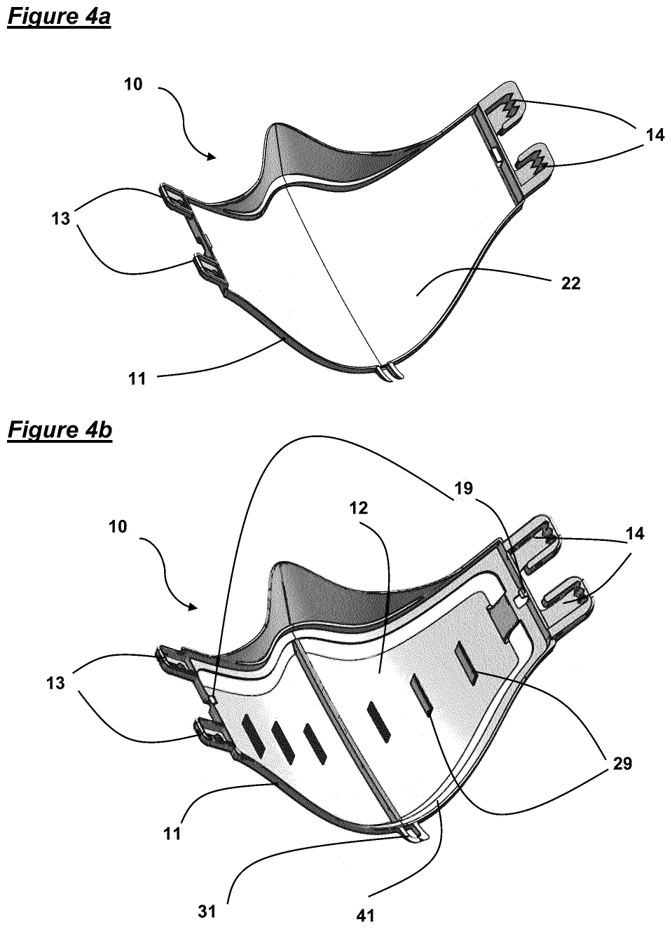

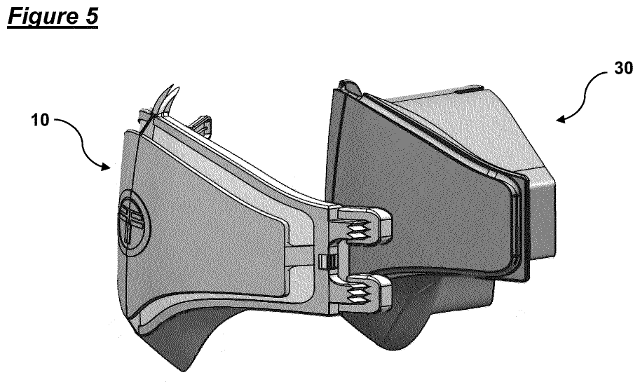

FIG. 4a is a perspective view of the filter located within the support frame of the first embodiment of a filter mask according to the invention during manufacture;

30 FIG. 4b is the view of FIG. 4a, without the filter;

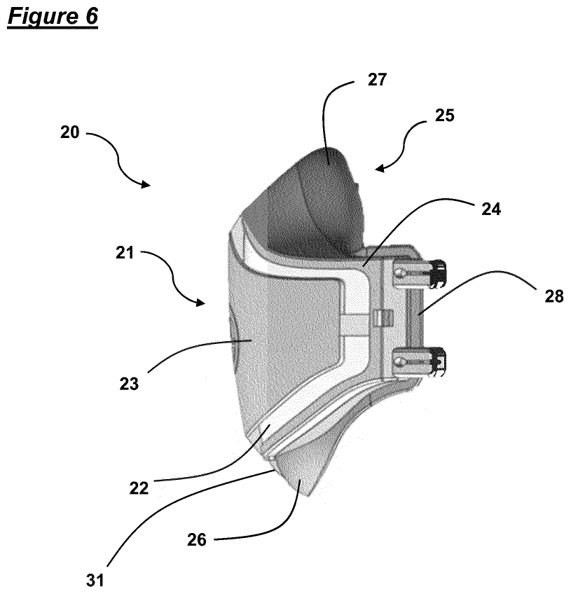

FIG. 5 is a perspective view of the support frame and filter of FIG. 4 and a core of the mould using during manufacture of the first embodiment of a filter mask according to the invention;

FIG. 6 is a side view of the first embodiment of a filter mask according to the invention;

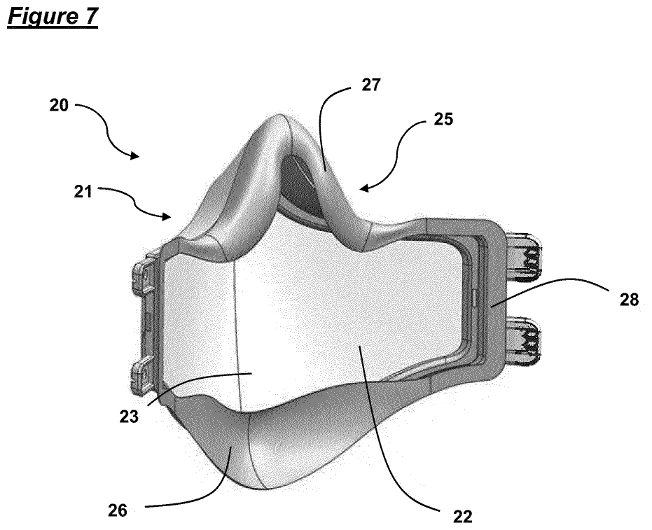

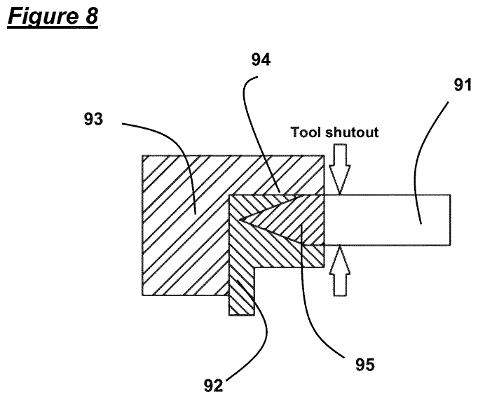

FIG. 7 is a perspective view of the rear of the first embodiment of a filter mask according to the invention;

FIG. 8 is a schematic drawing showing the connection between the support frame and the filter material formed by overmoulding of the sealing member;

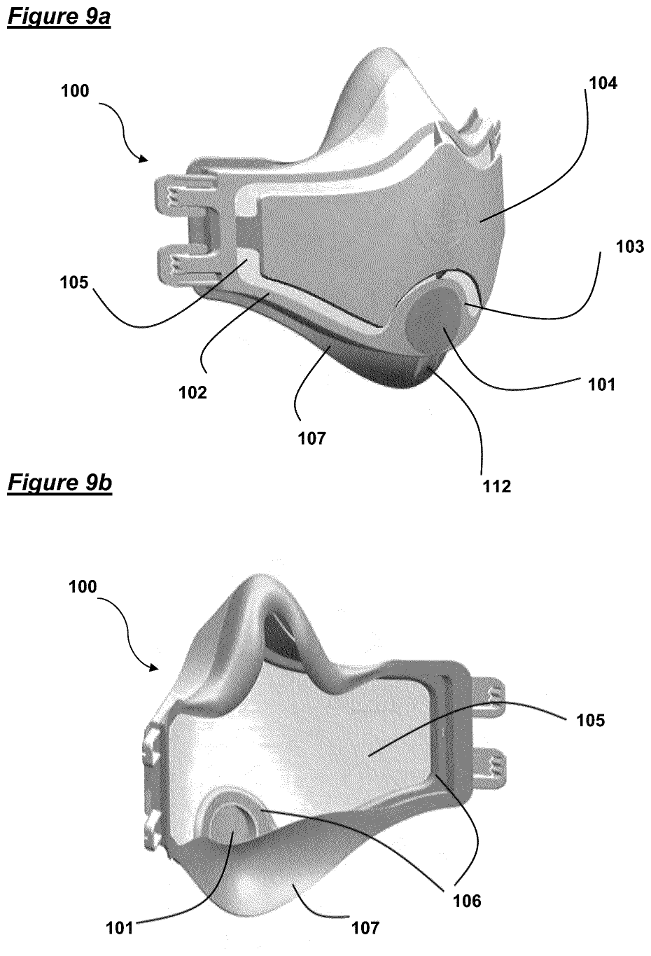

FIGS. 9a and 9b are perspective views of the front and rear of a further embodiment of a filter mask according to the invention;

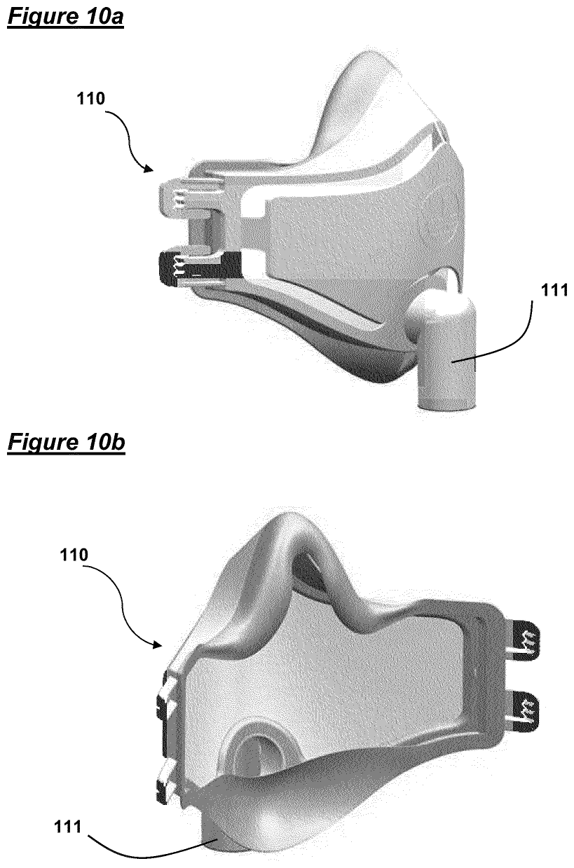

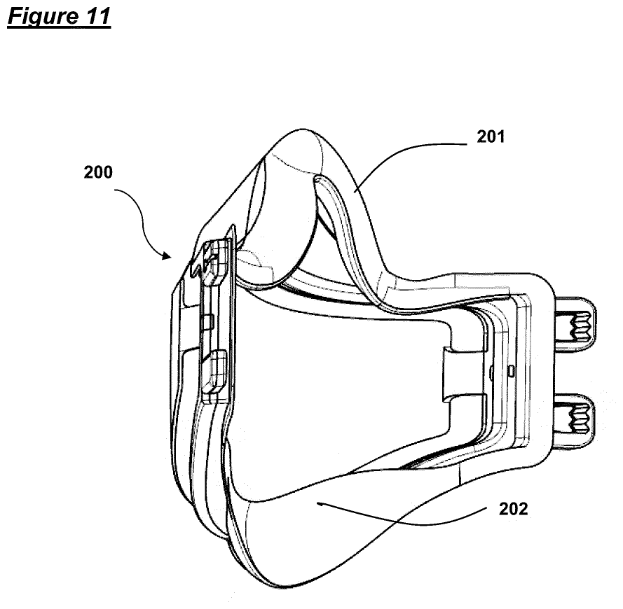

FIGS. 10a and 10b are perspective views of the front and rear of a further embodiment of a filter mask according to the invention;

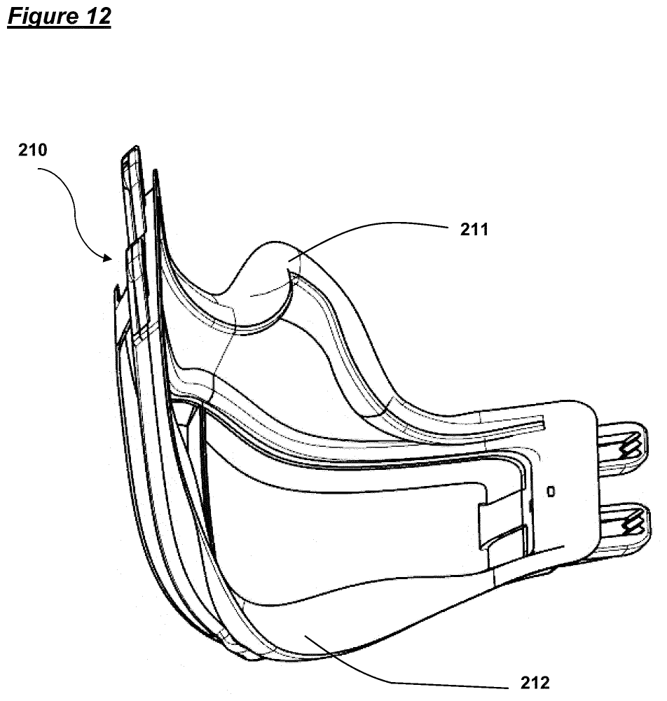

FIGS. 11 and 12 are perspective views of the rear of a face mask according to a further embodiment of the invention;

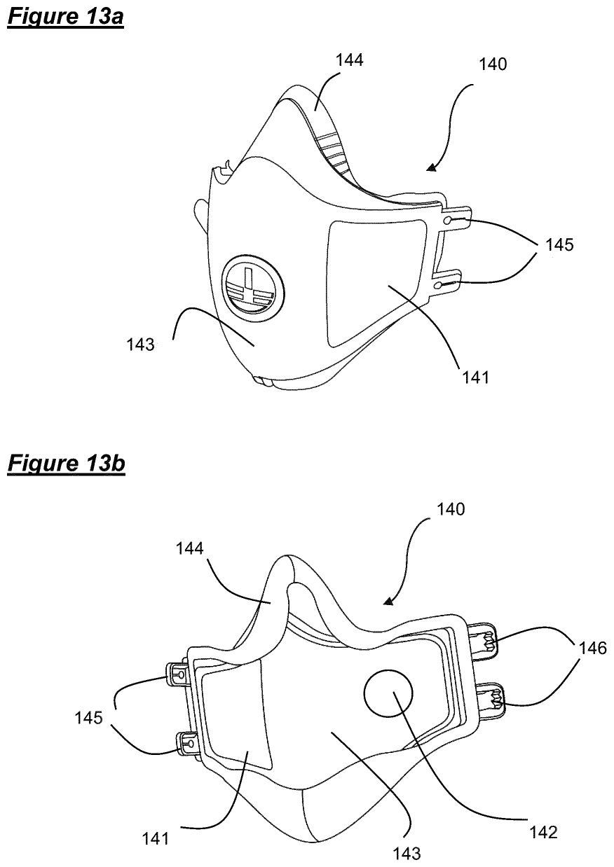

FIGS. 13a and 13b show front and rear perspective views of a filter mask 25 according to a further embodiment of the invention;

A support frame 10 for use in a filter mask according to the invention is shown in FIGS. 1 and 2. The support frame 10 has a generally curved shape and comprises an integrally formed rim 11, a shield 12, a pair of first attachment 30 formations 13 and a pair of second attachment formations 14. The shield 12 is substantially the same shape as the rim 11, but is smaller, and is offset from the rim 11 by approximately 1 cm. The shield 12 is attached to the rim 11 by four connection members 16 equally spaced about the circumference of the rim, and attached to points close to the edge of the shield. A space 15 between the shield 12 and the rim 11 extends about the periphery of the shield, interrupted only by the connection members 16. A seal supporting member 31 extends downwardly from the centre of the bottom edge of the rim 11. The seal supporting member 31 is an arcuate shaped tab which depends downwardly from the rim 11 and curves towards the rear of the support frame 10. The support frame 10 is an integrally formed component, with all features manufactured in a single injection moulding step.

A pair of first attachment formations 13 outwardly extend from a first side of the rim 11 which, in use, is located near a first ear of the wearer. The first attachment formations 13 take the form of a keyhole shaped slot through which a cord or strap (not shown) may be threaded, and secured with a knot or clamping device. A pair of second attachment formations 14 extend outwardly from a second side of the rim which, in use, is located near the second ear of the wearer. The second attachment formations 14 comprise a slot (not visible) covered by a tab 17. The tab 17 has a series of teeth 18 which provide grip. The cord or strap (not shown) passes through the slot in the attachment formation 14 from the internal side of the support frame to the external side, between the tab 17 and an edge of the slot. The teeth 18 grip the cord and reduce or prevent movement of the cord in the reverse direction.

In use, the cord or strap is secured in the upper first attachment formation 13, passes through the upper second attachment formation 14 from the internal side to the external side, through the lower second attachment formation 14 from the external side to the internal side, and is finally secured in the lower first attachment formation 13. Thus, the strap passes behind the wearer's head twice, with a loop being formed between the pair of second attachment formations 14 on the external side of the support frame. When the wearer applies tension to this loop, eg by pulling the loop, the cord will be pulled through the apertures, past the teeth 18 on the tab 17. The release of this tension eg by the wearer releasing the loop of cord, will allow the teeth 18 to grip the cord, thus preventing the cord from sliding back through the slot unless a significant force is applied. In this way, one hand may be easily used to adjust the mask to fit the wearer's head.

Injection moulding is used to produce the support frame 10, the support frame 10 being an integrally formed unit produced from injection moulded polypropylene in a single step.

Where the manufacture of the filter mask is automated, the support frame 10 is ejected from the mould onto a vacuum cup on a robot arm. A pneumatic die pushes a sheet of filter material 22 from a roll of filtration material into the support frame 10, the roll of filter material comprising a continuous series of pre-stamped sheets of filter material, heat sealed about their peripheral edges. The sheet of filter material 22 is shown in FIG. 3.

The sheet of filter material is positioned on the internal side of the support frame that is, the side of the support frame 10 which will face the wearer in use. The sheet of filtering material is located on the inwardly extending shoulder, held in place about its periphery by retention tabs 19 at either side of the rim. This support frame/filter material assembly is shown in FIGS. 4a and 4b.

The support frame/filtration material assembly is transferred into a first half of the mould and mounted to a mould core 30. A second half of the mould is sealed against the first half of the mould, forming the mould cavity. In an injection moulding step, a sealing member 25 is overmoulded to the support frame/filtration material assembly, surrounding the periphery of the sheet of filter material 22 and adhering it to the support frame 10, and forming the sealing member 25, in a single overmoulding step. The filter mask 20 is removed from the mould, eg using a large 20 mm pin to unpeel the filter mask 20 from the core 30, and transferred for manual attachment of an elasticated cord and subsequent packaging.

The connection between the filter, sealing member and support frame is shown schematically in FIG. 8. The periphery of the filter 91 is placed against a shoulder 94 extending laterally from the rim of the support frame 93, either before or after the support frame is placed in the mould. A thermoplastic elastomer (TPE) 92 is injected into the mould to form the sealing member. The portion of TPE 92 shown in FIG. 8 represents only a proximal edge of the sealing member. The TPE 92 is forced by the mould between the peripheral edge of the filter 91 and the rim and shoulder of the support frame 93, 94. The TPE 92 surrounds and impregnates the peripheral edge of the filter 91, the peripheral, impregnated region being denoted 95, adhering it to the rim and shoulder of the support frame 93, 94. Tool shutout, as shown in FIG. 8, prevents ingress of TPE 92 into the filter 91 beyond its periphery 95, ensuring that the filtering action of the filter 91 is not inhibited by TPE. As well as aiding adhesion of the support frame 93, 94 to the filter, impregnation of the peripheral edge 95 of the filter 91 with TPE may contribute to the seals at the edge of the filter, formed by heat sealing, preventing moisture or other contaminants from seeping into the structure of the filter.

A side view of a filtration mask 20 according to the invention is shown in FIG. 6.

The support frame 21 is the same as that described in relation to FIGS. 1 and 2. A sheet of filtering material 22 is positioned behind the support frame 21, and is visible in the gap between the shield 23 and the rim 24, about the periphery of the shield 23. This permits air to easily flow past the shield 23 and through the filtering material 22, while still benefitting from the protection of the shield across the filtering material.

The sealing member 25 is overmoulded onto the support frame 21 and filter material 22, fixing their relative positions. The sealing member 25 comprises a chin engaging portion 26, a nose engaging portion 27 and cheek engaging portions 28. The cheek engaging portions 28 extend laterally across the cheeks, forming a seal against the softer skin on the cheek and improving the overall seal of the mask.

The sealing member 25 is formed of an elastomeric material. It provides a compressible region between the support frame 21 and the wearer's face, enabling the elastomeric material to conform to the contours of the wearer's face and provide a good seal against it.

The downwardly depending seal supporting member 31 abuts the seal in the chin region, reinforcing the seal in this region and enhancing the seal against the wearer's chin.

FIGS. 4a and 4b show the support frame/filter member assembly, with and without the filter material present respectively. The support frame 10 comprises a rim 11. The rim 11 has an inwardly extending shoulder 41 onto which a pre-cut sheet of filter material 22 is placed. Extending from a first side of the rim are first attachment formations 13, and second attachment formations 14 extend from the second side of the rim. The support frame 10 also comprises a shield 12. A series of vertical ribs 29 across the back of the shield 12 ensure that the spacing between the shield 12 and the filter material 22 is maintained.

A further embodiment of a filter mask according to the invention can be seen in FIGS. 9a and 9b, and is generally designated 100. The structure of the mask is substantially the same as that described in relation to the first embodiment depicted in FIGS. 6 and 7, except that the mask 100 further comprises a mushroom valve 101 (shown in the closed position). In the chin region, the rim 102 of the support frame comprises an upwardly projecting portion 103, and the shape of the shield 104 and filter material 105 corresponds to the shape of the inner periphery of the rim 102 such that they accommodate the upwardly projecting portion 103. The filter material 105 is sealed along the periphery of the rim 102 of the support frame, including the upwardly projecting portion 103, by a surrounding rim of TPE 106. The upwardly projection portion comprises an aperture, with the mushroom valve 101 being formed in the aperture as part of the overmoulding step which also forms the sealing member 107. The mushroom valve 101 permits exhaled air to escape without passing through the filter material 105. The mushroom valve 101 could alternatively be accommodated within the shield 104.

A further embodiment of a filter mask shown in FIGS. 10a and 10b, which is generally denoted 110, has substantially the same structure is described in the second embodiment in relation to FIGS. 9a and 9b, but shows an elbow connection 111 in place of the mushroom valve. The elbow connection 111 permits connection of the mask 110 to other medical equipment, such as a nebuliser.

A further embodiment of a face mask is shown in FIGS. 11 and 12. These figures show filter masks 200, 210 in which the seal has been at least partially manufactured by gas-assisted injection moulding such that, in the nasal region 201, 211 the sealing member comprises an internal chamber at least partially bounded by a resiliently deformable enclosing wall, the enclosing wall including an external surface, the external surface having a form that is determined by the cavity of the mould. The sealing member in the chin region 202, 212 comprises both an inwardly and outwardly depending lip portion relative to the peripheral edge of the mask body.

FIGS. 13a and 13b depict a filter mask 140 comprising a filter 141, a gas inlet 142, a mask body 143, a sealing member 144 depending from a periphery of the mask body 143, and first and second pairs of attachment members 145, 146, which are as described in relation to FIGS. 4a-b. The gas inlet 142 may be a spigot. An aperture in a first side of the mask body 143 houses the filter 141, while the gas inlet 142 is inserted through or integrally formed with a second side of the mask body 143, such that the filter 141 and gas inlet 142 are located on different sides of the wearer's nose and mouth, in use. During use, oxygen flows from the gas inlet 142 to the filter 141, washing over the wearer's mouth and nose, providing a continuous flow of oxygen and helping to draw away exhaled gases.

1. A method of manufacturing a sealing member for use in a filter mask, the method comprising the steps of:
(a) providing a mould having a cavity, a polymer injection port and a gas inlet port;
(b) injecting a polymer through the polymer injection port into the cavity of the mould; and
(c) introducing gas through the gas inlet port into the cavity of the mould,
thereby forming a sealing member of the filter mask, wherein the sealing member of the filter mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user.

2. A method of manufacturing at least a portion of a sealing member for use in a filter mask, the method comprising the steps of:
(a) providing a mould having a cavity, a polymer injection port and a gas inlet port;
(b) injecting a polymer and a blowing agent through the polymer injection port into the cavity of the mould; and
(c) introducing gas through the gas inlet port into the cavity of the mould, to form a sealing member,
wherein the sealing member comprises an internal chamber at least partially bounded by a resiliently deformable enclosing wall formed of the polymer, the enclosing wall including an external surface, the external surface having a form that is determined by the cavity of the mould.

3. The method of claim 2, wherein the blowing agent is a physical blowing agent.

4. A method of manufacturing a filter mask, the filter mask comprising a support frame, a filter and a sealing member, wherein the method comprises:
a) locating the filter and the support frame in a mould; and
b) forming the sealing member by:
i) providing a mould having a cavity, a polymer injection port and a gas inlet port;
ii) injecting a polymer through the polymer injection port into the cavity of the mould; and
iii) introducing gas through the gas inlet port into the cavity of the mould, thereby forming a sealing member of the filter mask, wherein at least part of the sealing member of the filter mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user;
and wherein the sealing member is brought into engagement with the filter and the support frame, during injection moulding of the sealing member, in a manner that fixes the sealing member to the support frame and the sheet of filtering material.

5. The method of claim 4, wherein a blowing agent may be injected with the polymer in step ii).

6. A sealing member for a filter mask, at least a portion of the sealing member comprising an internal chamber at least partially bounded by a resiliently deformable enclosing wall, the enclosing wall including an external surface, the external surface having a form that is determined by the cavity of the mould.

7. A sealing member for a filter mask, at least a portion of the sealing member comprising an internal chamber at least partially bounded by a resiliently deformable enclosing wall, the enclosing wall including a face-contacting surface, the enclosing wall having a plurality of gas pockets formed therein.

8. A sealing member according to claim 7, wherein the face-contacting surface provides an anatomical fit with a user.

9. The method of claim 4 or claim 5, or the sealing member of any of claims 6 to 8, wherein only the nasal portion of the sealing member comprises an internal chamber at least partially bounded by a resiliently deformable enclosing wall.

10. A method of manufacturing at least a portion of a sealing member for use in a respiratory mask, the method comprising the steps of:
(a) providing a mould having a cavity, a polymer injection port and a gas inlet port;
(b) injecting a polymer through the polymer injection port into the cavity of the mould; and
(c) introducing gas through the gas inlet port into the cavity of the mould,
thereby forming at least a portion of a sealing member of the respiratory mask, wherein the at least a portion of the sealing member of the respiratory mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user, at least in the nasal region.

11. The method of claim 10, wherein the sealing member of the respiratory mask comprises an internal chamber at least partially bounded by a resiliently deformable enclosing wall formed of the polymer, the enclosing wall including a face-contacting surface, the face-contacting surface having a form that is determined by the cavity of the mould and provides an anatomical fit with a user, only in the nasal region.

12. A method of manufacturing at least a portion of a sealing member for use in a respiratory mask, the method comprising the steps of:
(a) providing a mould having a cavity, a polymer injection port and a gas inlet port;
(b) injecting a polymer and a blowing agent through the polymer injection port into the cavity of the mould; and
(c) introducing gas through the gas inlet port into the cavity of the mould, to form a sealing member,
wherein at least the nasal region of the sealing member comprises an internal chamber at least partially bounded by a resiliently deformable enclosing wall formed of the polymer, the enclosing wall including an external surface, the external surface having a form that is determined by the cavity of the mould.

13. The method of claim 12, wherein only the nasal region of the sealing member comprises an internal chamber at least partially bounded by a resiliently deformable enclosing wall formed of the polymer, the enclosing wall including an external surface, the external surface having a form that is determined by the cavity of the mould.

14. The method of claim 12, wherein the blowing agent is a physical blowing agent.

15. A sealing member for a respiratory mask, at least a portion of the sealing member comprising an internal chamber at least partially bounded by a resiliently deformable enclosing wall, the enclosing wall including an external surface, the external surface having a form that is determined by the cavity of the mould.

16. A sealing member for a respiratory mask, at least a portion of the sealing member comprising an internal chamber at least partially bounded by a resiliently deformable enclosing wall, the enclosing wall including a face-contacting surface, the enclosing wall having a plurality of gas pockets formed therein.

17. A sealing member according to claim 16, wherein the face-contacting surface provides an anatomical fit with a user.

18. A sealing member according to claim 15 or claim 16, wherein only the nasal portion of the sealing member comprises an internal chamber at least partially bounded by a resiliently deformable enclosing wall.
